(19) United States
(12) Patent Application Publication
(10) Pub. No.: US 2023/0167034 A1
(43) Pub. Date: Jun. 1, 2023

(54) SANDBLASTING SURFACE TREATMENT METHOD TO PREVENT SUBSURFACE DAMAGE OF THREE TYPES OF DENTAL ZIRCONIA AND INDUCE COMPRESSIVE STRESS THROUGH PHASE CHANGE

(71) Applicant: AJOU UNIVERSITY INDUSTRY-ACADEMIC COOPERATION FOUNDATION, Gyeonggi-do (KR)

(72) Inventor: Hee-Kyung KIM, Seoul (KR)

(73) Assignee: AJOU UNIVERSITY INDUSTRY-ACADEMIC COOPERATION FOUNDATION, Gyeonggi-do (KR)

(21) Appl. No.: 18/071,440

(22) Filed: Nov. 29, 2022

(30) Foreign Application Priority Data
Nov. 30, 2021 (KR) 10-2021-0169407

(51) Int. Cl. C04B 41/53; C04B 35/48; C04B 41/91; A61C 3/025; B24C 1/10; B24C 11/00

(52) U.S. Cl. CPC C04B 41/5346; C04B 35/48; C04B 41/91; A61C 3/025; B24C 1/10; B24C 11/00; C04B 2235/3246; A61C 13/083

(57) ABSTRACT

Provided is a surface treatment method for dental zirconia, which includes sandblasting the surfaces of three types of dental zirconia (3Y-TZP, 4Y-PSZ and 5Y-PSZ) with alumina particles, and when sandblasting conditions are optimized for each type of zirconia, the microstructure destruction of a subsurface layer may be minimized and compressive stress may be reinforced by a phase change, thereby improving mechanical properties, and the penetration of resin cement through microcracks inhibits crack propagation and thus is advantageous in increasing bonding efficiency of dental zirconia. In addition, a dental article including dental zirconia made by the surface treatment method for zirconia, and clinically suitable sandblasting protocols are provided.

### CROSS-REFERENCE TO RELATED APPLICATION

This application claims priority to and the benefit of Korean Patent Application No. 2021-0169407, filed on Nov. 30, 2021, the disclosure of which is incorporated herein by reference in its entirety.

### BACKGROUND

### 1. Field of the Invention

The present invention relates to a surface treatment method for a zirconia material, and more particularly, to a surface treatment method for minimizing the destruction of the microstructure of a subsurface layer and inducing compressive stress through a phase change.

### 2. Discussion of Related Art

Since a 3 mol % Y₂O₃-doped zirconia ceramic (3 mol % yttria-stabilized tetragonal zirconia polycrystal; 3Y-TZP) is used for a variety of critical uses such as crowns, root canal posts, orthodontic brackets and dental implants, it plays a critical role in dentistry. In addition, improvements in the zirconia ceramic manufacturing process combined with computer-aided design/computer-aided manufacturing (CAD/CAM) technology can lead to repeatable fabrication of customized dental prostheses with high accuracy. One of the main reasons for the considerable interest in 3Y-TZP in dentistry is that 3Y-TZP has the highest mechanical strength among ceramic oxides. Unlike other ceramics, zirconia is a metastable ceramic formed in a monoclinic, tetragonal, or cubic structure depending on a temperature. The excellent mechanical properties of zirconia ceramics result from the stress-induced strain strengthening mechanism around a crack tip.

Although zirconia ceramics exhibit excellent fracture toughness and strength, their inherent opacity often does not satisfy the aesthetic needs of a patient. The higher the yttria content (4 or 5 mol % partially stabilized zirconia; 4Y-PSZ or 5Y-PSZ), the larger the amount of isotropic cubic phase, and thus the translucency is greatly improved due to reduced light scattering in grains. Since the tetragonal-to-monoclinic phase transformation under tension is a major factor in determining fracture toughness, the mechanical strength of translucent zirconia may be damaged due to the limited amount of metastable tetragonal phase. With improved technology, zirconia ceramics are now preferable dental restorative materials, replacing metal-based restorations. However, in the aspect of clinical durability, one of the major concerns with zirconia restorations is that they have a problem in bonding with resin cement due to their chemically inactive nature and hardness. Particularly, abrasive blasting generally known as sandblasting has been implemented to roughen a zirconia surface that is hard and easily broken for better adhesion.

When a brittle material is impacted by a hard particle, plastic deformation occurs on the entire surface of a crack tip and thus compressive and shear stresses are generated, resulting in radial cracks just below the impact region. After the yield point, the tensional residual stress remaining in the material upon loading may cause lateral cracks that lead to material removal, and the characteristics of sandblasting particles such as a hardness, a size and a shape may affect kinetic energy as a result of particle-target interaction. The kinetic energy of blasting particles is decreased in particle diameter from 670 μm to 420 μm, which is a 3.5-fold decrease, reducing the plastic deformation of the surface. Therefore, the effect of hit may depend on a particle mass. Essentially, larger blasting particles cause morphological defects on the zirconia surface. According to several studies, sandblasting can cause phase transformation of 3Y-TZP and improve flexural strength, whereas severe sandblasting conditions lower the mechanical strength of 3Y-TZP, resulting in an excessive monoclinic phase content.

Recently, the adhesive bonding behavior of high-transparent monolithic dental zirconia was investigated, confirming that high-translucent zirconia (5Y-PSZ) had a lower bonding strength to resin cement than conventional zirconia (3Y-TZP), but the bonding strength of high-transparent zirconia is greatly improved by mechanical sandblasting.

However, the optimal protocols for sandblasting parameters for high-translucent zirconia have not been established yet, and with regard to newly introduced high-translucent zirconia, there are currently no detailed studies on a subsurface change after sandblasting.

### SUMMARY OF THE INVENTION

As a result of trying to study the optimal protocols by considering durability in bonding between resin cement and high-translucent zirconia and a subsurface change after sandblasting, the inventors evaluated phase transformation and subsurface change after sandblasting, focusing on the effects of various Al₂O₃ particles on three different types of dental zirconia (3Y-TZP, 4Y-PSZ and 5Y-PSZ) while maintaining other parameters constant, and suggested the clinically optimal size and conditions for alumina particles for sandblasting.

Accordingly, the present invention is directed to providing a surface treatment method for dental zirconia, which includes: (a) polishing the surface of zirconia which includes mostly tetragonal and cubic zirconia with less than 15% monoclinic system, in which 95 vol % or more of all particles have an average diameter of 100 to 1200 nm, and which have a density of 99.5% or more of the theoretical density and are opalescent; and (b) sandblasting the polished surface in (a) with alumina particles using a nozzle-equipped sandblasting apparatus.

The present invention is also directed to providing a dental article which includes dental zirconia made by the surface treatment method for dental zirconia.

The terms used herein should not be interpreted as only explaining the present invention, not limiting it. Singular expressions include plural referents unless the context clearly indicates otherwise. The terms “include” and “have” used herein designate the presence of characteristics, numbers, steps, operations, components or members described in the specification or a combination thereof, and it should be understood that the possibility of the presence or addition of one or more other characteristics, numbers, steps, operations, components, members or a combination thereof is not excluded in advance. All terms including technical and scientific terms have the same meaning that is generally understood by those skilled in the art unless defined otherwise. General terms, such as terms defined in dictionaries, should be interpreted with meanings according to the context of related technology, and should not be interpreted with ideal or excessively formal meanings unless clearly defined herein.

The present invention is directed to providing a surface treatment method for dental zirconia, which includes: (a) polishing the surface of zirconia which includes mostly tetragonal and cubic zirconia with less than 15% monoclinic system, in which 95 vol % or more of all particles have an average diameter of 100 to 1200 nm, and which have a density of 99.5% or more of the theoretical density and are opalescent; and (b) sandblasting the polished surface in (a) with alumina particles using a nozzle-equipped sandblasting apparatus.

In one embodiment of the present invention, the zirconia in (a) may be any one selected from the group consisting of 3 mol % yttria-stabilized tetragonal zirconia polycrystal (3Y-TZP), 4 mol % partially stabilized zirconia (4Y-PSZ), and 5 mol % partially stabilized zirconia (5Y-PSZ).

In one embodiment of the present invention, in (b), the vertical distance between the nozzle-equipped sandblasting apparatus and the polished surface may be 1 to 100 mm.

In one embodiment of the present invention, in (b), the pressure for sandblasting with alumina particles may be 0.1 to 0.5 Mpa.

In one embodiment of the present invention, when the zirconia is 3Y-TZP, the average particle size of the alumina particles may be 100 to 120 μm.

In one embodiment of the present invention, when the zirconia is 3Y-TZP and the surface treatment method is performed with the alumina particles having an average particle size of 100 to 120 μm, a transformed layer with a depth of 2.0 to 3.0 μm may be induced.

In one embodiment of the present invention, when the zirconia is 4Y-PSZ, the average particle size of the alumina particles may be 40 to 60 μm.

In one embodiment of the present invention, when the zirconia is 4Y-PSZ, and the surface treatment method is performed with the alumina particles having an average particle size of 40 to 60 μm, a transformed layer with a depth of 0.2 to 1.2 μm may be induced.

In one embodiment of the present invention, when the zirconia is 5Y-PSZ, the average particle size of the alumina particles may be 40 to 60 μm.

In one embodiment of the present invention, when the zirconia is 5Y-PSZ, and the surface treatment method is performed with the alumina particles having an average particle size of 40 to 60 μm, a transformed layer with a depth of 0.2 to 1.2 μm may be induced.

In one embodiment of the present invention, the surface treatment method may be to prevent the crack growth and microstructure destruction of a subsurface layer and induce compressive stress due to a phase change.

The present invention provides a dental article which includes dental zirconia made by the surface treatment method for dental zirconia.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 is the schematic diagram of a sandblasting process; alumina particles are accelerated toward a specimen with a high pressure air stream through a circulator nozzle (diameter: 2.0 mm). The alumina particles collide with the specimen at a maximum velocity of 157 m/s;

FIG. 2 shows a model of a point indentation microfracture pattern. A plastic transformed region may be formed just below particle collision, and radial and lateral cracks start from the plastic processing region;

FIG. 3 shows the collision velocity of single abrasive particles, estimated according to a particle velocity measured by Jafar et al. V: velocity (m/s); x: particle size (μm);

FIG. 4 shows the X-ray diffraction (XRD) patterns of subgroups sandblasted with alumina particles having various sizes:

for 3 mol % yttria-stabilized tetragonal zirconia polycrystal (3Y-TZP): magnified graphs in (a) a 2θ range of 20 to 90° and (b) 27.5<2θ<30.5 and 58.5<2θ <60.5;

for 4 mol % partially stabilized zirconia (4Y-PSZ): magnified graphs in (c) a 2θ range of 20 to 90° and (d) 27.5<2θ<30.5 and 58.5<2θ<60; and

for 5 mol % partially stabilized zirconia (5Y-PSZ): magnified graphs in (e) a 2θ range of 20 to 90° and (f) 27.5<2θ<30.5 and 58.5<2θ<60.5, and a control had tetragonal and cubic phases. After sandblasting, the appearance of a monoclinic peak (−111) at 2θ=28.2° and the appearance of a rhombohedral peak (12-1) at 2θ=29.88 were confirmed with three zirconia grades (m: monoclinic; t: tetragonal; c: cubic; r: rhombohedral);

FIG. 5 shows the phase fraction values (wt %) determined by Rietveld analyses of XRD patterns for (a) a 3Y subgroup, (b) a 4Y subgroup and (c) a 5Y subgroup;

FIG. 6 shows Williamson-Hall plots of β cos θ with respect to 4 sin θ calculated from the XRD spectra for the (a) 3Y subgroup, (b) 4Y subgroup and (c) 5Y subgroup;

FIG. 7 shows the FIB cross-sections of (a) 3Ycon, (b) 3Y25, (c) 3Y125 and (d) 3Y110, and the thin boxes shown in (d) indicate locations of detailed information, wherein (e) indicates the detailed information on the transformed region of 3Y110 and (f) indicates the detailed information on the untransformed region of 3Y110;

FIG. 8 shows the FIB cross-sections of (a) 4Y50, (b) 4Y90 and (c) 4Y110, and the thin box in (c) indicates the detailed location (d) of the transformed region of the 4Y110;

FIG. 9 shows the FIB cross-sections of (a) 5Y25, (b) 5Y90, (c) 5Y110 with the thin box indicating the detailed location (d) of 5Y110, and (e) 5Y125 with the thin box indicating the detailed location (f) of 5Y125;

FIG. 10 shows the average depth of the tetragonal-to-monoclinic phase transformed regions obtained in the FIB/SEM images. Bilateral ANOVA shows that there is a statistically significant interaction between a zirconia grade with respect to the transformed region depth and the effect of abrasive particle size (p<0.05);

FIG. 11 shows schematic diagrams of the hemispherical stress fields against load in (a): 3Y125, (b): 4Y125 and (c): 5Y125 models, and (d) the impact stress fields in these models; and

FIG. 12 shows the thicknesses of stress layers affected by the finite element modeling for all zirconia grades and material cuts below the impact region.

### DETAILED DESCRIPTION OF EXEMPLARY EMBODIMENTS

As described above, the necessity for finding the optimal protocols by considering durability in bonding between resin cement and high-translucent zirconia and a subsurface change after sandblasting has emerged. The inventors quantified the subsurface change and crystal strain/stress state caused by phase transformation after sandblasting using X-ray diffraction (XRD) and Rietveld analyses. The extent of a subsurface change was determined by focused ion beam nanotomography (FIB-nt) according to a non-destructive continuous slicing procedure capable of observing first few microns below the surface. The effect of various kinetic energies (alumina particle sizes) on a subsurface change of three different dental zirconia substrates was confirmed using commercially available Al₂O₃ blasting particles with five different sizes. 3D finite element analysis (FEA) was performed to provide deep residual stress distribution in a sandblasted zirconia component. Microstructural and crystallographic changes in subsurface and residual stresses, caused by Al₂O₃ sandblasting, were evaluated with three different grades of dental zirconia with five different particle sizes. The subsurface change induced by sandblasting can be explained from the emergence of a new phase (rectangular rhomboid), the presence of microcracks, crack propagation or material removal, and compressive/tensile forces derived from the interaction between blasting media and a substrate surface. The present invention provides clinical guidelines for selecting optimal sandblasting particles to minimize the microstructure destruction of a subsurface layer and maximize compressive stress due to a phase change in a novel dental zirconia material.

Hereinafter, the present invention will be described in further detail.

The present invention provides a surface treatment method for dental zirconia, which includes: (a) polishing the surface of zirconia which includes mostly tetragonal and cubic zirconia with less than 15% monoclinic system, in which 95 vol % or more of all particles have an average diameter of 100 to 1200 nm, and which have a density of 99.5% or more of the theoretical density and are opalescent; and (b) sandblasting the polished surface in (a) with alumina particles using a nozzle-equipped sandblasting apparatus.

In one embodiment of the present invention, the zirconia in (a) may be any one from the group consisting of 3Y-TZP, 4Y-PSZ and 5Y-PSZ.

The dental zirconia used in the present invention can be used as an esthetic restoration material, and the dental zirconia shows physical properties within the above range.

Preferably, when the zirconia in (a) is 3Y-TZP, it may have <15% cubic systems; when the zirconia in (a) is 4Y-PSZ, it may have >25% cubic systems; and when the zirconia in (a) is 5Y-PS, it may have >50% cubic systems.

According to an exemplary embodiment of the present invention, in (b), the vertical distance between the nozzle-equipped sandblasting apparatus and the polished surface may be 1 to 100 mm, and preferably 5 to 20 mm. When the vertical distance is outside the above range, a surface roughness may not be properly induced, or compressive stress may not be sufficiently induced.

In one embodiment of the present invention, in (b), a pressure for sandblasting alumina particles may be 0.1 to 0.5 Mpa. When the pressure is outside the above pressures range, the dental zirconia may be more susceptible to subsurface damage, such as subsurface lateral cracking and plastic deformation.

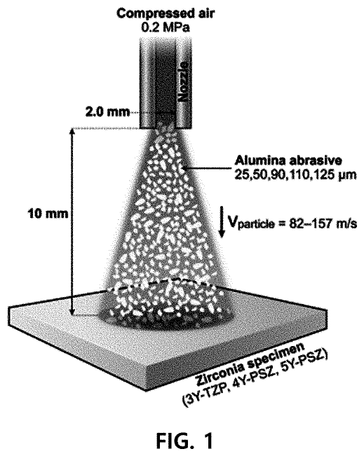

Specifically, in one embodiment of the present invention, as shown in FIG. 1, using the sandblasting apparatus, a zirconia plate was particle-polished 10 mm apart from the tip of the sandblasting apparatus at an impact angle of 90° under pressure of 0.2 MPa at 10 s/cm².

As a result, within the range of the treatment parameters investigated in the present invention, the mechanical properties were not degraded due to high susceptibility to surface damage, and the particle sizes optimized for realizing compressive stress-induced phase transformation were different from each other.

In one embodiment of the present invention, as shown in FIG. 5, in all zirconia grades, the higher the alumina particle size, the larger the amount of rhombohedral phase (up to 64.38 wt %), and the smaller the amount of tetragonal phase. As a result of the Rietveld analysis, a larger amount of such rhombohedral phase was observed in high-transparent zirconia, and therefore it is inferred that it is derived from a regular-tetrahedral phase and a regular-hexahedral phase. The occurrence of rhombohedral phase after polishing of 3Y-TZP ceramics may cause subsurface damage due to a volume increase caused by particle extraction, and the weak mechanical property of the sandblasted high-translucent zirconia may be derived from the formation of rhombohedral phase. In the present invention, the FIB cross-sectional image shows that there is a monoclinic phase gradient up to a depth of 2.9 μm for the 3Y subgroup (see FIG. 7). 3Y110 showed the largest compressive residual stress in the Williamson-Hall plot by inducing the deepest transformed layer with the smallest rhombohedral phase amount without formation of microcracks within the 3Y subgroup (FIG. 6).

As seen in the FEA model of the present invention, although the compressive residual stress induced by t-m strain can serve as the main driving force for lateral cracks and contribute to the strengthening mechanism, tensile stress is generated along the surface when a load is applied. In 3Y125, small subsurface microcracks were observed, reflecting the high stress concentration at the zirconia grain boundary. However, the subsurface tensile component may be large enough to undergo the transition of the material removal mode from ductility to brittleness in a blasting process. When critical tension build-up is reached and thus destruction begins in a zirconia material, grains collapse and material defects increase, thereby reducing flexural strength. When the particle size is less than 110 μm, the plastic deformation mechanism is activated and crack propagation is inhibited.

Using high-transparent zirconia ceramics, deformed monoclinic symmetry was created directly on the surface during sandblasting, and for 4Y50 of the 4Y subgroup, the maximum alumina particle size was 0.83 μm, while for 5Y25 of the 5Y subgroup, the maximum alumina particle size was 0.77 μm. When the alumina particle size increased, sandblasting did not induce a monoclinic transformed layer of isolated particles, but rather induced an amorphous transformed layer in translucent zirconia. Connection cracks occur between pores and migrate to the surface, causing material removal with larger alumina particles, and thus may be disadvantageous to mechanical properties. The rhombohedral phase amount increased up to 64.38 wt % for the 4Y subgroup, and up to 57.01 wt % for the 5Y subgroup (FIG. 5). The detection of a larger amount of rhombohedral phase in the 4Y subgroup resulted from the presence of a transformable square phase. A larger amount of a transformed rhombohedral phase in the 4Y subgroup results from the t-*r conversion, regardless of whether the rhombohedral phase is derived from the cubic or tetragonal phase.

The Williamson-Hall analysis identified in the present invention showed that a compressive lattice strain was not induced in high-translucent zirconia during sandblasting. In the 5Y subgroup, when the particle size increased, severe peak broadening without periodicity of crystallinity produced a scattering profile due to the presence of polycrystalline aggregates. Such an amorphous transformed layer was also confirmed in FIB images (FIGS. 7 to 9). It is known that an increased yttria content reduces crack propagation resistance of dental zirconia. In the present invention, the 5Y subgroup is more susceptible to subsurface damage such as subsurface lateral cracks, plastic deformation and surface melting. Surface melting and abnormal grain growth found in 5Y125 may be due to a localized high temperature. During sandblasting, the particle kinetic energy is converted to thermal energy, causing local melting of the surface of the dental zirconia ceramic. It has been reported that grain pull-out and surface deterioration are promoted at high temperatures, and an increased grain size can promote crack formation.

In the present invention, as mechanical tests for confirming crystallographic and morphological changes after sandblasting in three different zirconia grades, sandblasting was performed on the zirconia surface. In addition, numerical analysis by the finite element method was used to determine theoretical stress distribution and understand empirical data obtained from the mechanical tests. The FEA models in the 5Y subgroup showed a deeper erosive cut and a deeper affected stress layer (FIG. 11). In addition, the 5Y subgroup showed a smaller amount of the maximum principal stress than the 3Y or 4Y subgroup to reduce load-failure. A soft substrate absorbs a large amount of kinetic energy under an impact load, causing large plastic deformation. In the present invention, the FIB cross-sectional image of high-transparent zirconia with larger particles showed that abrasive particles penetrated the zirconia matrix in the form of brittle fracture due to the interaction between the alumina particles and the zirconia material. Larger particles are more susceptible to fractures since they can gain higher kinetic energy than smaller ones. Accordingly, when setting sandblasting protocols, it is necessary to consider a lower damage tolerance of high-translucent zirconia than that of conventional zirconia.

Sandblasting of high-transparent zirconia may reduce flexural strength, but may improve adhesion to resin cement due to microcracks generated during sandblasting, and therefore, a higher shear adhesive strength may compensate for an adverse effect on mechanical properties. In relation to tooth bonding, filling enamel microcracks using an adhesive resin may prevent crack propagation and increase fracture toughness.

The depth of the monoclinic transformation region may greatly affect the amount of compressive residual stress. As shown in FIG. 10, in the 3Y subgroup, larger particles increased compressive residual stress determined by Williamson-Hall analysis to create a transformed region to a depth of 1.1 to 2.9 μm below the surface. Unlike the 3Y subgroup, when the depths of 4Y and 5Y transformed regions were less than 1 μm, sandblasting did not cause compressive stress on the surface. The thickness of the transformed layer under stress applied from the outside may be too small to induce compressive stress. Surface microcracks may be advantageous to increasing bonding efficiency of dental zirconia by inhibiting crack propagation due to the penetration of resin cement through cracks. Therefore, a microcrack sealing mechanism may contribute to an increase in mechanical strength of the zirconia material. However, when there is a lateral crack in surface connection, it may have an adverse effect on the mechanical behavior of the zirconia system and long-term reliability of dental prostheses. In the embodiment of the present invention, the larger the alumina particles, the wider the lateral cracks at a few micrometers below the surface, but this is not deep enough to affect structural integrity in very highly translucent zirconia.

As a result, three grades of dental zirconia ceramics showed different sandblasting reactions for various sizes of alumina particles based on their inherent crystallographic and mechanical properties.

Accordingly, in one embodiment of the present invention, when the zirconia is 3Y-TZP, the average particle size of alumina particles may be 100 to 120 μm.

In one embodiment of the present invention, when the zirconia is 3Y-TZP, and the surface treatment method is performed with alumina particles having an average particle size of 100 to 120 μm, it can be characterized by inducing a transformed layer having a depth of 2.0 to 3.0 μm.

In one embodiment of the present invention, when the zirconia is 4Y-PSZ, the average particle size of alumina particles may be 40 to 60 μm.

In one embodiment of the present invention, when the zirconia is 4Y-PSZ, and the surface treatment method is performed with alumina particles having an average particle size of 40 to 60 μm, a transformed layer having a depth of 0.2 to 1.2 μm may be induced.

In one embodiment of the present invention, when the zirconia is 5Y-PSZ, the average particle size of alumina particles may be 40 to 60 μm.

In one embodiment of the present invention, when the zirconia is 5Y-PSZ, and the surface treatment method is performed with alumina particles having an average particle size of 40 to 60 μm, a transformed layer having a depth of 0.2 to 1.2 μm may be induced.

Specifically, as described in the embodiment of the present invention, to consider the effect of the size of alumina particles on latent subsurface damage and compressive stress induced from three zirconia grades and to prevent a significant decrease in mechanical strength, the vertical distance between a nozzle-equipped sandblasting apparatus and the polished surface is 1 to 100 mm, and sandblasting is performed under pressure of 0.1 to 0.5 MPa, and for 3Y-TZP, the alumina particle size is 100 to 120 μm, and for 4Y-PSZ or 5Y-PSZ, the alumina particle size is 40 to 60 μm. Outside the above range, mechanical properties may be degraded due to high susceptibility to surface damage under applied stress.

In one embodiment of the present invention, the surface treatment method may be a surface treatment method of dental zirconia preventing crack growth and the destruction of a microstructure of a subsurface layer and inducing compressive stress due to a phase change.

The present invention provides a dental article including dental zirconia made by the surface treatment method of dental zirconia. Dental zirconia may be further processed, using commercially available dental CAD/CAM systems, into dental articles, such as dental restorations (blanks, full-contour fixed partial dentures (FPDs), bridges, implant bridges, multi-unit frameworks, abutments, crowns, partial crowns, veneers, inlays, onlays, occlusal braces, orthodontic spacing devices, tooth replacement, splints, dentures, posts, teeth, jackets, facing, occlusal facets, implants, cylinders and connections.

Hereinafter, the present invention will be described in more detail with reference to examples. The examples are merely provided to more fully describe the present invention, and it will be obvious to those of ordinary skill in the art that the scope of the present invention is not limited to the following examples.

### Preparation Example

Conventional tetragonal zirconia (3Y-TZP; KATANA ML, Kuraray Noritake Dental, Tokyo, Japan) and next-generation highly translucent zirconia containing cubic phases (4Y-PSZ and 5Y-PSZ, respectively, KATANA STML and KATANA UTML, Kuraray Noritake Dental). Each grade (14.0 mm×14.0 mm×1.0 mm) of fully sintered plate-shaped zirconia specimens was polished with 400-, 600- and 800-grit silicon carbide papers, and thermally etched at 1400° C. for 30 minutes in air. Then, each grade of specimens (n=12 for each zirconia grade) was divided into 6 groups according to an alumina abrasive particle size. Five sizes of alumina particles (25, 50, 90, 110, and 125 μm; Cobra, Renfert GmbH, Hilzingen, Germany) were air-polished using a sandblasting apparatus (Basic master, Renfert) under pressure of 0.2 MPa at a distance of 10 mm from the specimen surface at 10 s/cm². A sample group of each grade, which was not subjected to sandblasting, was used as a control. Only the polished surface was sandblasted. A sandblasting process is schematically shown in FIG. 1.

### Example 1

### Evaluation of Phase Deformation and Compressive Strain—XRD Analysis

To determine a crystal structure and phase deformation, one specimen in each experimental subgroup of each zirconia grade was analyzed. Powder XRD measurement was performed using a graphite monochromator (λCuKα=0.15418 nm)-equipped DMAX-2200PC X-ray diffractometer (Rigaku, Tokyo, Japan). A step scan mode was used with a step size of 0.02° in the 2θ range of 20 to 90° for a counting time of 4 seconds for each step. Quantitative phase analysis was performed by the Rietveld refinement method using the Fullprof program, and intensity profiles were fitted using the pseudo-Voigt function.

The surface deformation/stress induced by sandblasting was evaluated by the Williamson Hall (W-H) method in a uniform deformation model (UDM). The physical extension of the XRD peak as a function of microstrain was considered according to Equation (1).

$$s {s}_{h k l} \cdot cos θ = ( \frac{K λ}{L} ) + ( 4 ε \cdot sin θ ) \qquad ( 1 )$$

Here, L indicates a nanocrystal size; K indicates a shape factor generally considered to be 0.89 for ceramic materials; λ indicates a radiation wavelength in nanometers; β indicates the peak full width at half maximum (radians); θ indicates the peak diffraction angle; and ε indicates a strain induced by crystal deformation.

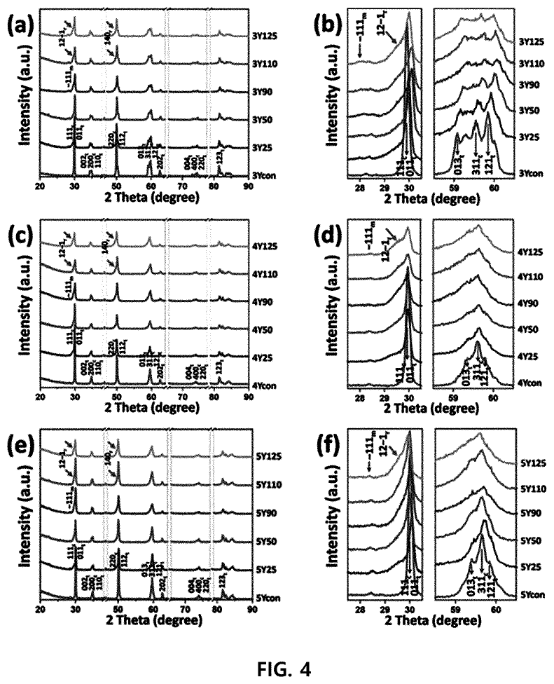

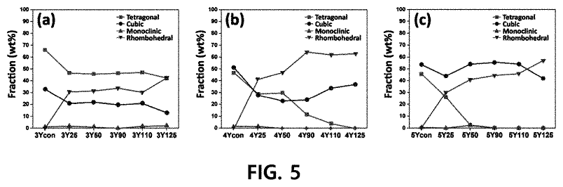

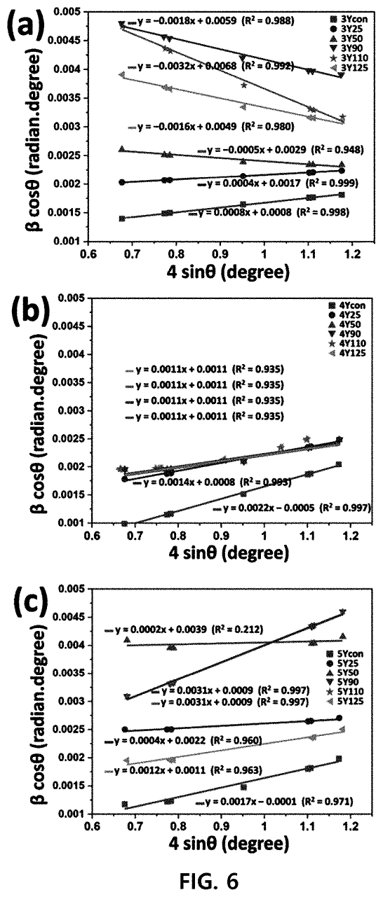

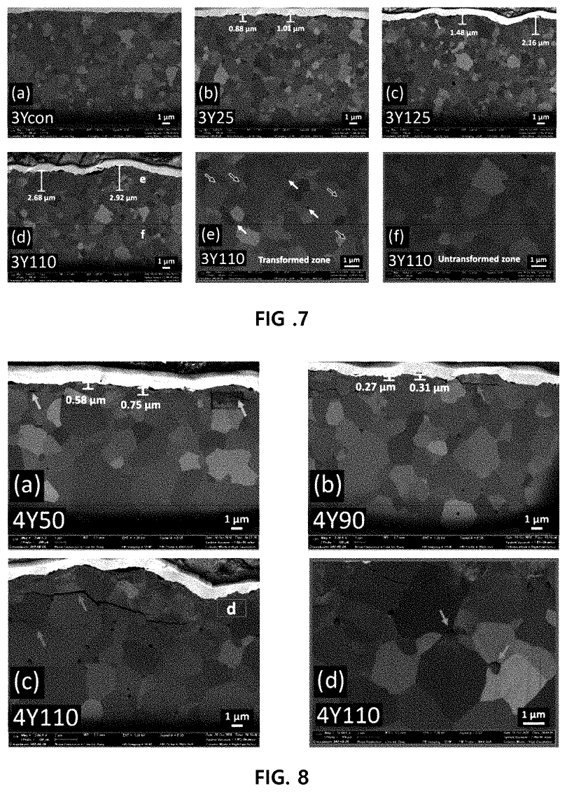

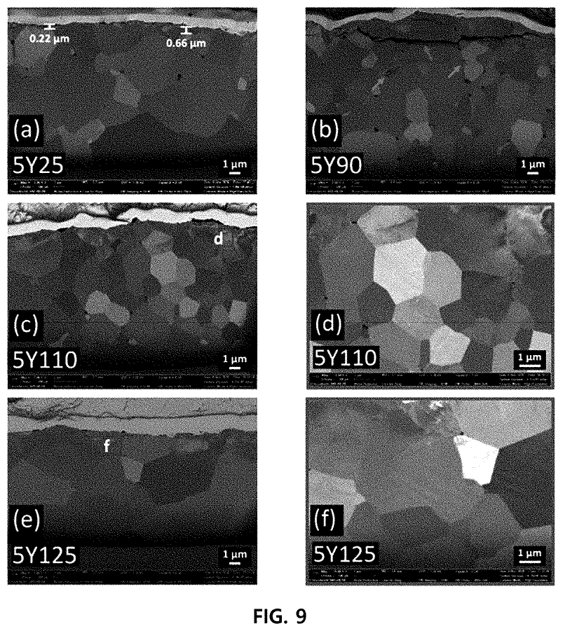

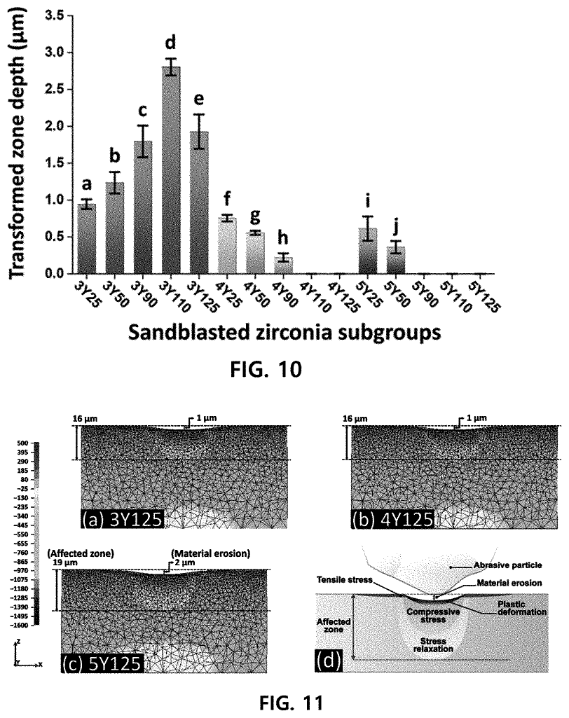

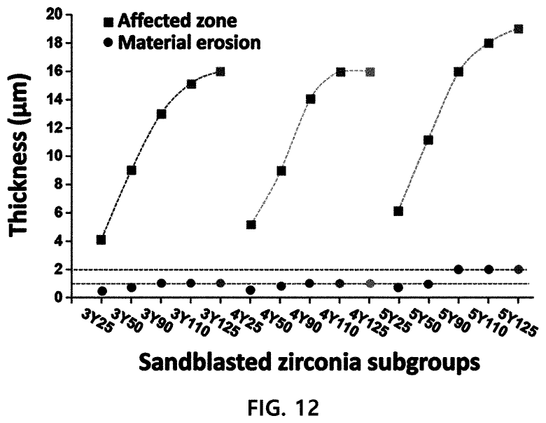

As a result, FIG. 4 showed XRD patterns for all subgroups of each zirconia grade and 2θ angle ranges of 27.5° to 30.5° and 58.5° to 60.5°. The 3Y subgroup is shown in (a) and (b); the 4Y subgroup is shown in (c) and (d); and the 5Y subgroup is shown in (e) and (f).

FIG. 5 shows the phase differentiation obtained by Rietveld micromodulation for (a) the 3Y subgroup, (b) the 4Y subgroup and (c) the 5Y subgroup. As shown in FIGS. 4 and 5, the control generally has two crystal structures (tetragonal and cubic), a cubic content increased as the Y₂O₃ content increased (for 3Ycon, 32.9 wt %, for 4Ycon, 51.4 wt %, and for 5Ycon, 53.7 wt %). The amount of monoclinic phase is considered negligible in the control. After sandblasting, specimens of each grade showed asymmetric peak widenings of (011)t at 2θ=30.27° and (112)t at 2θ=50.38°, and widened with the use of larger blasting particles. As shown in FIG. 4, the (011)t peak shift occurs at a higher angle due to sandblasting, and the maximum peak shift occurred in 3Y110 of the 3Y subgroup, 4Y110 of the 4Y subgroup, and 5Y25 of the 5Y subgroup. Generally, the smallest peak shift was observed in the 5Y subgroup among the three zirconia grades.

The sandblasting conditions used in this study generated only a small portion of monoclinic phase (0-2.3 wt %), and for the 3Y subgroup, 3Y125 showed the highest monoclinic phase content of 2.3 wt %, for the 4Y subgroup, 4Y25 showed the higher monoclinic phase content of 1.8 wt %, and for the 5Y subgroup, 5Y50 showed the highest monoclinic phase content of 2.18 wt %, and the monoclinic phase content in 4Y50, 4Y90, 4Y110, 4Y125, 5Y90, 5Y110 and 5Y125 was 0. After sandblasting, the rhombohedral phase (r-ZrO2) was confirmed at a low angle of the (011)t peak for all zirconia grades having the highest intensity (12-1)r peak at 2θ=29.88. FIG. 5 shows that in all zirconia grades, as the blasting particle size increased, the rhombohedral phase content increased (for the 4Y subgroup, the maximum 64.38 wt %, and for the 5Y subgroup, the maximum 57.01 wt %), whereas the cubic phase content decreased. For the 5Y subgroup, the cubic phase content was drastically reduced to 0 in 5Y90.

Characteristics related to sandblasting of the three zirconia grades using various sizes of Al₂O₃ were compared and plotted based on the assumption of the isotropic properties of crystals stressed from interplanar spacing by a modified form of W-H analysis and UDM (FIG. 6). As shown in FIG. 4, it was confirmed that the intensity of the tetragonal (011) peak at 2θ=30.27° was sharp and narrow in the control, and a polished specimen (control) of each zirconia grade showed high crystallinity. The strain-induced expansion of the square (011) peak caused by lattice deformation after sandblasting was calculated, and FIG. 6 showed a plot formed with 4 sin θ along the x axis, and β cos θ along the y axis. A linear function and a strain/stress fit to the experimental data for each subgroup were evaluated from the slope value. The 3Y110 plot showed the steepest negative slope, indicating the highest compressive strain/stress. A positive slope value is associated with a tensile strain/stress, and a negative value is associated with a compressive strain/stress. For the 4Y or 5Y subgroup, there was no line with a negative slope. For the 4Y subgroup, after 4Y50, there was no more change in slope. For the 5Y subgroup, after 5Y50, the low accuracy of the profile fitting and severe peak widening were observed.

Taken together, in the present invention, in the three dental zirconia grades, the larger the alumina particle size, the larger the rhombohedral phase amount (maximum 64.38 wt %), but the smaller the tetragonal phase amount. The 3Y110 plot showed the steepest negative slope, indicating the largest compressive strain/stress, and for the 4Y and 5Y subgroups, after 4Y50 and 5Y50, there were no changes in slope or the low accuracy of the profile fitting and severe peak widening were observed.

### Example 2

FIB/SEM Analyses

Microstructural changes in the near-surface region were observed in sandblasting with different specimens of each experimental subgroup of each zirconia grade. A thin layer of platinum (1 μm) was deposited on the specimen, and then a fragmented specimen was prepared using a focused ion beam (FIB; ZEISS CrossBeam 540, Carl Zeiss Microscopy GmbH, Oberkochen, Germany) milling device equipped with a Zeiss Capella FIB column and a Gemini II SEM column. The sandblasted surface was milled with Ga+ ions at 30 kV using an ion current sequence decreasing up to the final polishing step of 300 pA. The FIB/SEM images of each cross-section were obtained using an Energy-Selective Backscatter (EsB). The transformed region depth of each FIB cross-section was measured on 10 randomly selected sites using a line measurement tool for measuring the longitudinal distance of the image using ImageJ software (v1.53e, National Institutes of Health, Bethesda, Md., USA).

The cross-sectional FIB-SEM images of the sandblasted specimens are shown in FIGS. 7 to 9.

FIG. 7 shows the FIB cross-sections of (a) 3Ycon, (b) 3Y25, (c) 3Y125, and (d) 3Y110, wherein the thin boxes in (d) indicate the locations of detailed information (e and f). (e) is the detailed information on the transformed region of 3Y110, and (f) is the detailed information on the untransformed region of 3Y110. The transformed regions containing comb-patterned crystal grains were observed within several micrometers in the upper area below the surface. Specifically, in (e), the modified monoclinic particles with twin crystals are indicated by blue arrows, small pores are indicated by yellow arrows, no microcracks were detected, and no transformed particles were found in (f). For 3Y125, a small microcrack (indicated by the green arrow) was detected close to the surface. Such comb-patterned crystals are considered monoclinic grains generated during the tetragonal-to-monoclinic transformation by a martensitic twinning mechanism. For 3Y125, small interfacial microcracks that were not connected to each other were detected.

FIG. 8 shows the FIB cross-sections of (a) 4Y50, (b) 4Y90 and (c) 4Y110, wherein the thin box region in (c) indicates the detailed location (d) of the transformed region of 4Y110. There were microcracks (indicated by the green arrows) and their depths were first limited to 1 μm in 4Y50, but the microcracks grew in a horizontal direction to be connected to the surface in 4Y90. 4Y110 shows a uniform transformed layer instead of isolated transformed particles below the surface, and lateral cracks are located a maximum of 4.5 μm below the surface. When the cracks expand in a lateral direction, material removal (brittle crack) may occur. Specifically, (d) shows that the grain boundary is fractured, and plastic deformation is detected. Alumina particle debris (indicated by the orange arrows) was deposited below the surface.

FIG. 9 shows the FIB cross-sections of (a) 5Y25, (b) 5Y90, (c) 5Y110, with the thin box in (c) indicating the detailed location (d) of 5Y110, and (e) 5Y125, with the thin box in (e) indicating the detailed location (f) of 5Y125. In 5Y25, a thin transformed layer was found 0.7 μm below the surface, and in 5Y90, lateral cracks were connected to the surface where there were no separated transformed grain regions. Alumina particle debris (indicated by the orange arrows) was detected in 5Y90. In (d) and (f), uniform transformed regions having plastic deformation and surface melting were found. In 5Y125, abnormal particle growth was observed.

Taken together, unlike 3Y-TZP, in the 4Y or 5Y subgroup, cracks were connected to each other and such intergranular or transgranular cracks led to easier zirconia material removal during sandblasting as the particle size increased. The propagation of the microcracks progressed parallel to the surface, and in the 4Y or 5Y subgroup, such lateral cracking was located a maximum of 4.5 μm below the surface. The lateral crack expansion may generate material removal, causing brittle fracture. Compared to 4Y-PSZ, larger cracks occurred in 5Y-PSZ with smaller blast particles. There were no separated transformed regions below the surfaces of 4Y110 of the 4Y subgroup or 5Y90 of the 5Y subgroup, but uniform defect layers were found. In 5Y125, abnormal grain growth that can impair mechanical stability was observed.

FIG. 10 shows the degree of t-m phase transformation according to a depth direction in all zirconia subgroups. Two-way ANOVA was performed to examine the effects of a zirconia grade and an abrasive particle size on a transformed region depth. There was a statistically significant interaction between the effects of the zirconia grade and abrasive particle size on the transformed region depth (p<0.05). In the case of the 3Y subgroup, 3Y110 induced the deepest transformed layer with a maximum depth of 2.9 μm. In the case of the 4Y subgroup, 4Y25 induced the deepest transformed layer with a maximum depth of 0.8 μm. In the case of the 5Y subgroup, 5Y25 induced the deepest transformed layer of a maximum depth of 0.7 μm. However, in the case of the 4Y subgroup and the 5Y subgroup, no more transformed layers were detected from 4Y110 and 5Y90.

After polishing the 3Y-TZP ceramics, the generation of a rhombohedral phase may cause subsurface damage due to a volume increase caused by particle pull-out, and weak mechanical properties of the sandblasted high-translucent zirconia may result from the rhombohedral phase formation. In the present invention, the FIB cross-sectional image shows that there is a monoclinic phase gradient up to a depth of 2.9 μm for the 3Y subgroup. For 3Y110, microcracks are not formed in the 3Y subgroup and the rhombohedral phase amount induces the smallest and deepest transformed layer, resulting in the greatest compressive residual stress in the Williamson-Hall plot.

Unlike the 3Y subgroup, since 4Y and 5Y transformed region depths were less than 1 μm, sandblasting did not cause compressive stress on the surface. The thickness of the transformed layer under externally defined stress may be too small to cause compressive stress. Surface microcracks may be advantageous in increasing the bonding efficiency of dental zirconia by inhibiting crack propagation by penetrating resin cement through cracks. Therefore, a microcrack sealing mechanism may contribute to an increased mechanical strength of the zirconia material. However, lateral cracks in the surface connection may have an adverse effect on the mechanical behavior of the zirconia system and the long-term reliability of dental prostheses.

Taken together, the larger the alumina particles, the wider the lateral cracks at several micrometers below the surface, but the lateral cracks were not deep enough to affect structural integrity in the highly translucent zirconia. Thus, the size of the optimal alumina particles for sandblasting was different for each zirconia grade.

### Example 3

Interaction Between Polishing Particles and Zirconia Matrix

The stress field applied with single blasting particles was simulated by a finite element method (FEM) using LS-DYNA software (v10.0, Livermore Software Technology Corporation (LSTC), Livermore, Calif., USA). The material elastic properties of a 3D-FEA model are shown in Table 1.

TABLE 1

| | | | | | Young's | Flexural |
|---|---|---|---|---|---|---|
| | | Particle size | | Poisson's | modulus | strength |
| Material | | (μm) | Density | ratio | (GPa) | (MPa) |
| Zirconia | ML | 0.52 ± 0.05 | 6.10 | 0.30 | 210 | 800-900 |
| | (3Y-TZP) | | | | | |
| | STML | 1.19 ± 0.20 | 6.10 | 0.30 | 210 | 560-650 |
| | (4Y-PSZ) | | | | | |
| | UTML | 1.58 ± 0.17 | 6.10 | 0.30 | 210 | 470-500 |
| | (5Y-PSZ) | | | | | |
| Abrasive | Al₂O₃ | 25, 50, 90, 110, 125 | 3.98 | 0.22 | 375 | 379 |
| particle | | | | | | |

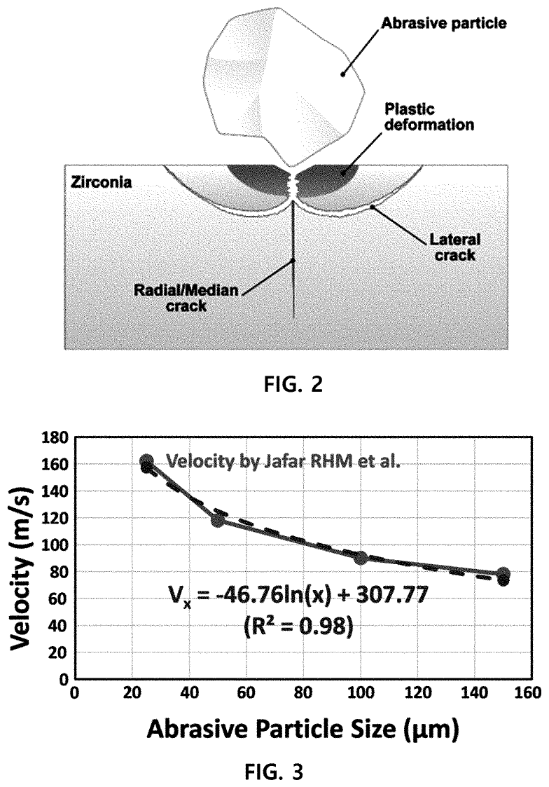

After constructing the model, a linear elastic analysis was performed under dynamic load. In the indentation model, it was assumed that the stress field affected under particle impact was hemispherically symmetric. Since ceramic materials can be damaged by brittle fracture, the maximum principal stress (MPS) was considered to evaluate the stress applied to the load under the impact region. The analysis of the tensile (positive) or compressive (negative) stress in impact was performed on the zirconia components in all models. The contact between the abrasive particles and the zirconia matrix was represented by a simplified model (FIG. 2) of a point indentation microcrack pattern as a function of alumina particle size. A material erosive region was calculated by adaptive meshing and element deletion. The meshing process was performed on a model with a 10-node quadratic tetrahedral element, and the mesh size was set to 0.001 mm According to the data reported by Jafar et al., the collision velocity of single abrasive particles may be estimated and thus is calculated according to Equation (2) below.

Vx=−46.761n(x)+307.77(R²=0.98) (2)

Here, V is a velocity (m/s), and x is a particle size (μm). V₂₅=157 m/s; V₅₀=125 m/s; V₉₀=97 m/s; V₁₁₀=88 m/s; and V₁₂₅=82 m/s. The finite element model used in this study is based on the hypothesis that all materials are linear-elastic and homogeneous under stress, and have no defects in their components.

The hemispherical maximum main stress fields with respect to load in the representative models of 3Y125, 4Y125 and 5Y125 are shown in FIG. 11. The compressive stress (negative) was concentrated in the impact region and the tensile stress (positive) was relieved by approaching the surface. The affected region near the blasted surface is divided into 4 regions: (i) a plastic transformed region that is several micrometers deep having microstructure transformation to some degree below material erosion; (ii) a tensile stress region where tensile stress is generated on the surface; (iii) a compressive stress region in which residual compressive stress is induced under particle impact; and (iv) a stress relaxation region in which residual stress is partially relieved. The 5Y125 model had a deeper erosive cut than the 3Y125 or 4Y125 model with a depth of 2 μm. Below the material erosion, a plastic transformed region was developed. In the 5Y125 model, a deeper stress relaxation region with a depth of 19 μm was shown compared to the 3Y125 or 4Y125 model. The maximum tensile/compressive stress (MPa) introduced into the model design is shown in Table 2. The result shows that the finite element model of the 5Y subgroup has a lower MPS value than the 3Y or 4Y subgroup.

TABLE 2

| | | |
|---|---|---|
| | Maximum principal | Maximum principal |
| Model | tensile stress | compressive stress |
| design | (positive values) | (negative values) |

| | | | |
|---|---|---|---|
| 3Y | 3Y25 | 416.06 | 1533.85 |
| | 3Y50 | 402.89 | 1542.26 |
| | 3Y90 | 480.76 | 1460.81 |
| | 3Y110 | 425.25 | 1416.71 |
| | 3Y125 | 397.41 | 1576.75 |
| 4Y | 4Y25 | 412.96 | 1543.43 |
| | 4Y50 | 402.98 | 1554.53 |
| | 4Y90 | 479.02 | 1472.94 |
| | 4Y110 | 430.63 | 1423.44 |
| | 4Y125 | 399.69 | 1570.46 |
| 5Y | 5Y25 | 402.63 | 1397.91 |
| | 5Y50 | 399.91 | 1408.50 |
| | 5Y90 | 397.71 | 1308.88 |
| | 5Y110 | 376.20 | 1240.51 |
| | 5Y125 | 336.36 | 1306.07 |

The thicknesses of stress layers affected by the finite element modeling for all zirconia grades and material cuts below the impact region are shown in FIG. 12. The material erosion depth in the particle impact region did not exceed 1 μm in the case of the 3Y or 4Y subgroup model, and reached a maximum of 2 μm in the case of the 5Y subgroup model. Larger particles induced a more deeply affected stress layer for all zirconia grades. The models of the 5Y subgroup showed deeper stress dissipation compared to the models of the 3Y or 4Y subgroup.

Taken together, three grades of dental zirconia ceramics showed different sandblasting reactions with respect to various alumina particle sizes caused by inherent crystallographic and mechanical properties. To consider the effect of the size of alumina particles on latent subsurface damage and compressive stress induced from three zirconia grades and to prevent a significant decrease in mechanical strength, the recommended alumina particle sizes are 110 μm for 3Y-TZP, and 50 μm for 4Y-PSZ or 5Y-PSZ.

In the present invention, the crystallographic and microstructural subsurface changes were evaluated on three different dental zirconia grades after sandblasting with alumina having five different particle sizes.

(1) In conventional zirconia, alumina sandblasting induced tetragonal-to monoclinic phase transformation, but the phase transformation depends on a metastable tetragonal phase amount in high-transparent zirconia.

(2) Care should be taken not to inhibit mechanical properties due to high susceptibility to surface damage under stress applied when selecting an appropriate sandblasting protocol for high-transparent zirconia.

(3) Within the range of treatment parameters investigated in the present invention, the recommended conditions for realizing compressive stress-induced phase deformation without significant subsurface damage include sandblasting particles with a size of 110 μm for 3Y-TZP, or 50 μm for 4Y-PSZ or 5Y-PSZ, a pressure of 0.2 MPa, a distance of 10 mm apart from a specimen surface, and a velocity of 10 s/cm².

Statistical Analysis

Statistical analysis was performed using software (IBM SPSS Statistics for Windows, v25.0, IBM Corp., Chicago, Ill., USA) satisfying the significant level a=0.05. The Shapiro-Wilk test was performed to evaluate the normal distribution, and the Levene test was applied to test the homogeneity of the variances. The averages of the transformed region depth according to the crystal structure were compared between experimental subgroups. Two-way analysis of variances (ANOVA) was applied to analyze the effects of a zirconia grade and an abrasive particle size on the transformed region depth after sandblasting.

When a surface treatment method according to the present invention is used, it can minimize the microstructure destruction of a subsurface layer through sandblasting and induce compressive stress caused by a phase change, thereby improving mechanical properties, and is advantageous in increasing the bonding efficiency of dental zirconia by inhibiting crack propagation by the penetration of resin cement through microcracks. Accordingly, the bonding strength can be increased to reduce the repair rate due to peeling and structural destruction even after the procedure. In addition, for three types of dental zirconia, since the optimal size and conditions of alumina particles for sandblasting are presented, a clinically preferable surface treatment method can be provided.

It will be apparent to those skilled in the art that various modifications can be made to the above-described exemplary embodiments of the present invention without departing from the spirit or scope of the invention. Thus, it is intended that the present invention covers all such modifications provided they come within the scope of the appended claims and their equivalents.

What is claimed is:

1. A surface treatment method for dental zirconia, comprising:
(a) polishing the surface of zirconia which includes mostly tetragonal and cubic zirconia with less than 15% monoclinic system, in which 95 vol % or more of all particles have an average diameter of 100 to 1200 nm, and which have a density of 99.5% or more of the theoretical density and are opalescent; and
(b) sandblasting the polished surface in (a) with alumina particles using a nozzle-equipped sandblasting apparatus.

2. The method of claim 1, wherein the zirconia in (a) is any one selected from the group consisting of 3 mol % yttria-stabilized tetragonal zirconia polycrystal (3Y-TZP), 4 mol % partially stabilized zirconia (4Y-PSZ), and 5 mol % partially stabilized zirconia (5Y-PSZ).

3. The method of claim 1, wherein, in (b), the vertical distance between the nozzle-equipped sandblasting apparatus and the polished surface is 1 to 100 mm.

4. The method of claim 1, wherein, in (b), the pressure for sandblasting with alumina particles is 0.1 to 0.5 Mpa.

5. The method of claim 1, wherein, when the zirconia is 3Y-TZP, the average size of the alumina particles is 100 to 120 μm.

6. The method of claim 5, wherein the surface treatment method is performed to induce a transformed layer with a depth of 2.0 to 3.0 μm.

7. The method of claim 1, wherein, when the zirconia is 4Y-PSZ, the average particle size of the alumina particles is 40 to 60 μm.

8. The method of claim 7, wherein the surface treatment method is performed to induce a transformed layer with a depth of 0.2 to 1.2 μm.

9. The method of claim 1, wherein, when the zirconia is 5Y-PSZ, the average particle size of the alumina particles is 40 to 60 μm.

10. The method of claim 9, wherein the surface treatment method is performed to induce a transformed layer with a depth of 0.2 to 1.2 μm.

11. The method of claim 1, wherein the surface treatment method prevents the crack growth and microstructure destruction of a subsurface layer and induces compressive stress due to a phase change.

12. A dental article comprising dental zirconia made by the surface treatment method for zirconia of claim 1.
